(19) United States
(12) Patent Application Publication
(10) Pub. No.: US 2026/0212569 A1
(43) Pub. Date: Jul. 23, 2026

(54) SIMULATED PHOTON-COUNTING COMPUTED TOMOGRAPHY

(71) Applicant: Siemens Medical Solutions USA, Inc., Malvern, PA (US)

(72) Inventors: Ziad Burbar, Knoxville, TN (US); Inki Hong, Knoxville, TN (US)

(21) Appl. No.: 19/032,171

(22) Filed: Jan. 20, 2025

(51) Int. Cl. G06T 11/00; G06T 7/30; G16H 30/40

(52) U.S. Cl. CPC G06T 12/10; G06T 7/30; G16H 30/40; G06T 2207/10081; G06T 2207/10104

(57) ABSTRACT

Systems and methods include generation of a first image volume based on electrical signals output by energy-integrating photon detectors, input of the first image volume to a network trained to generate a simulated photon-counting CT image from an energy-integrating CT image, reception of a first simulated photon-counting CT image generated by the network in response to the input first image volume, and presentation of the first simulated photon-counting CT image on the display.

### BACKGROUND

Conventional Computed Tomography (CT) detectors form images by integrating received energy. Specifically, these detectors receive photons into a scintillation crystal which absorbs the photons and converts them into visible light. A photodiode attached to the backside of each detector cell converts the light into an electrical signal. The detector cells are separated by reflective septa to prevent light crosstalk between the cells. The electrical signal integrates all of the light converted from all photons which are received by the photodiode during an integration time. Since all of the received light is integrated into one electrical signal, the spectral information of the individual photons received during the integration time is lost.

Photon-counting CT detectors, in contrast, directly transform photons into electrical signals. A semiconductor absorbs a received photon, which creates an electron-hole pair in the semiconductor. An electric field is maintained between a cathode disposed on a photon-receiving side of the semiconductor and pixelated anodes on an opposite side of the semiconductor. The electric field separates the electron-hole pair, causing current which specifically corresponds to the received photon to flow through an anode.

Photon-counting CT detectors exhibit several advantages compared to energy-integrating detectors. Individual detector cells are defined by the electric field between the common cathode and the pixelated anodes, so there is no need for additional septa between the detector pixels to prevent optical crosstalk inherent to energy-integrating detectors. The geometrical dose efficiency and spatial resolution of photon-counting CT detectors is therefore greater than that of energy-integrating detectors.

The signal-to-noise ratio of photon-counting CT detectors is also higher than that of energy-integrating detectors. Since the electrical signals generated by an energy-integrating detector are necessarily unfiltered to ensure that all detected light is integrated into the signals, these electrical signals also include low energy electronic noise inherent to the system. Photon-counting CT detectors are able to filter out the electronic noise to result in individual electrical signals corresponding to each received photon. By improving the signal-to-noise ratio, radiation dose may be reduced while maintaining image quality.

Moreover, a photon-counting CT detector exhibits intrinsic spectral sensitivity by detecting the signal peaks created by individual photons as well as their respective energy levels. Multiple energy thresholds may be used to differentiate tissues based on their composition and attenuation properties at different energy levels. This differentiation allows for improved distinction between tissues which have similar photon densities. Precisely capturing the energy of each photon also improves quantification of specific tissue properties, such as attenuation coefficients or iodine concentrations in contrast-enhanced scans. The improved differentiation and quantification can result in better diagnosis and treatment planning.

Photon-counting CT detectors are not yet readily available to most clinicians and researchers. Systems are desired to obtain some of the benefits provided by photon-counting CT detectors using energy-integrating CT detectors.

### BRIEF DESCRIPTION OF THE DRAWINGS

FIG. 1 is a schematic diagram of an energy-integrating CT detector.

FIG. 2 is a schematic diagram of a photon-counting CT detector according to some embodiments.

FIG. 3 is a block diagram of a system to generate a simulated photon-counting CT image according to some embodiments.

FIG. 4 is a block diagram of a system to generate a photon emission tomography (PET) image based on a simulated photon-counting CT image according to some embodiments.

FIG. 5 is a flow diagram of a process to train a network to generate a simulated photon-counting CT image according to some embodiments.

FIG. 6 illustrates training of a neural network to generate a simulated photon-counting CT image according to some embodiments.

FIG. 7 is a block diagram of a system to generate a PET image based on a simulated photon-counting CT linear attenuation coefficient map according to some embodiments.

FIG. 8 is a flow diagram of a process to train a network to generate a simulated photon-counting CT linear attenuation coefficient map according to some embodiments.

FIG. 9 illustrates training of a neural network to generate a simulated photon-counting CT linear attenuation coefficient map according to some embodiments.

FIG. 10 illustrates training of a neural network to generate a simulated photon-counting CT image and a simulated photon-counting CT linear attenuation coefficient map from an integral CT image according to some embodiments.

FIG. 11 is a block diagram of an imaging system according to some embodiments.

### DETAILED DESCRIPTION

The following description is provided to enable any person in the art to make and use the described embodiments. Various modifications, however, will remain apparent to those in the art.

Embodiments utilize a trained neural network to derive a simulated photon-counting CT image from an energy-integrating CT image. The simulated photon-counting CT image may provide improved spatial resolution, signal-to-noise ratio, tissue differentiation and/or quantification as compared to the energy-integrating CT image.

Embodiments may also or alternatively generate a simulated photon-counting CT image-derived linear attenuation coefficient map from an energy-integrating CT image. The map may be used to reconstruct a PET image from PET data which was acquired contemporaneously to the energy-integrating CT image. The linear attenuation coefficients of the linear attenuation coefficient map may be more accurate than coefficients of a hypothetical linear attenuation coefficient map derived from the energy-integrating CT image. Accordingly, the quality of the resulting PET image may be higher than that of a PET image reconstructed from the PET data and a linear attenuation coefficient map derived from the energy-integrating CT image.

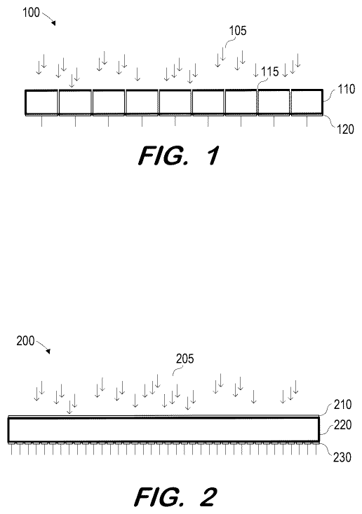

FIG. 1 is a schematic diagram of a portion of energy-integrating CT detector 100. Scintillation crystal 110 may comprise thallium-doped sodium iodide or any other suitable material. Septa 115 divide crystal 110 into cells and a photodiode 120 is attached to each cell. Scintillation crystal 110 receives photons 105 emitted by an X-ray tube, typically after the photons have passed through collimation elements (not shown). Each cell absorbs the photons 105 it receives and converts the photons 105 into visible light. The photodiode 120 of each cell converts the visible light within the cell into an electrical signal which corresponds to the cell. All the electrical signals generated by photodiodes 120 during a time period (i.e., an integration time) are used to generate a two-dimensional projection image. For example, each cell represents an image pixel, and the electrical signal corresponding to a cell determines a value (e.g., a number of Hounsfield Units) assigned to the image pixel.

FIG. 2 is a schematic diagram of photon-counting CT detector 200 according to some embodiments. Photons 205 pass through cathode 210 and are absorbed by semiconductor 220 (e.g., cadmium telluride). The absorption creates electron-hole pairs and the electrons are directed to anode electrodes 230 by an electric field established between cathode 210 and electrodes 230. Each 230 represents an image pixel and receives individual electrical charges of each photon received at the pixel. A signal processing component generates a projection image based on the charges created by individual photons at each pixel and their energy levels. The signal processing component may also set a threshold energy below which electrode signals are ignored, ensuring that the only signals generated from valid photons are considered and that the resulting projection image exhibits a high signal-to-noise ratio.

Embodiments encompass all manner of photon-counting CT detectors that are or become known. Such detectors may be alternatively referred to as spectral CT detectors, silicon-based photon-counting CT detectors, photon-counting detectors, and photon counters, for example.

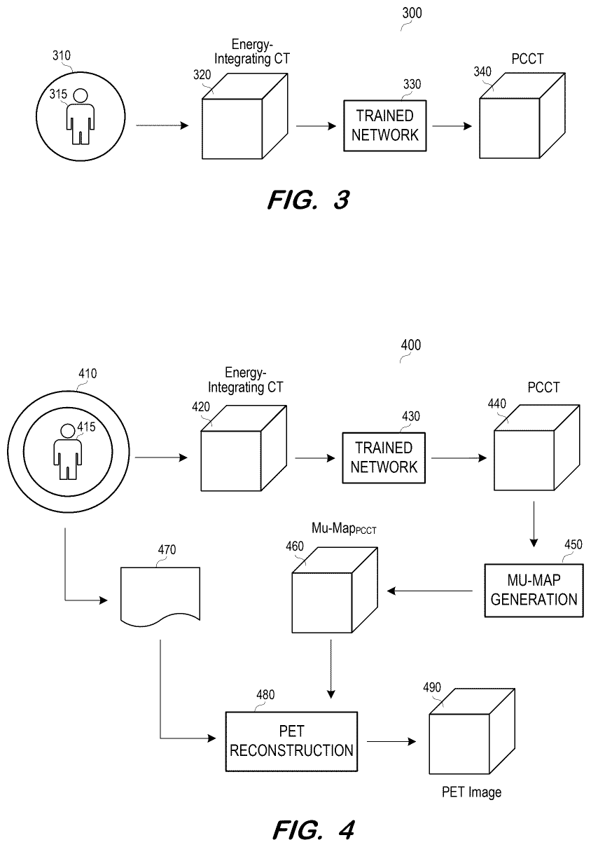

FIG. 3 is a block diagram of system 300 to generate a simulated photon-counting CT image according to some embodiments. All system components described herein may be implemented in computer hardware, in program code and/or in one or more computing systems executing such program code as is known in the art. Such a computing system may include one or more processing units which execute program code stored in a memory system. More than one functional component may be implemented by a single computing system in some embodiments. One or more of the computing systems may comprise a virtual machine, and one-or more computing systems may comprise a cloud-based compute resource providing on-demand scalability and failure recovery.

CT scanner 310 uses an energy-integrating CT detector to acquire CT data. The CT data includes a set of data acquired at each of projection angles P₀ to Pₙ with respect to patient 315 while an X-ray tube and the energy-integrating CT detector are positioned at the various projection angles. CT scanner 310 applies conventional CT reconstruction algorithms to the acquired CT data to generate energy-integrating CT image 320.

Energy-integrating CT image 320 is input to trained neural network 330, which operates according to its training to generate simulated photon-counting CT image 340. Network 330 may comprise any supervised or unsupervised learning-compatible network to receive image data and to output image data that is or becomes known. For example, network 330 may comprise a generator of a Generative Adversarial Network (GAN) having trainable parameters as is known in the art. Simulated photon-counting CT image 340 may exit better spatial resolution, signal-to-noise ratio, tissue differentiation and/or quantification than energy-integrating CT image 320.

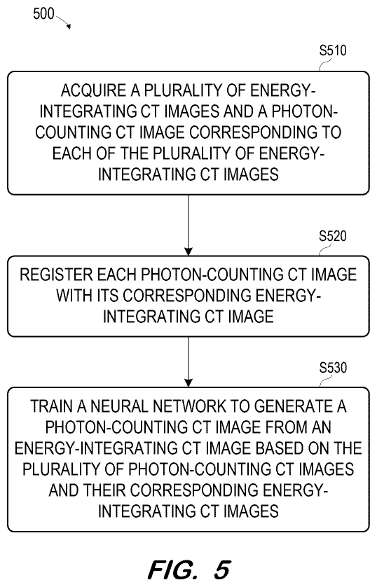

FIG. 4 is a block diagram of system 400 to generate a PET image based on a simulated photon-counting CT image according to some embodiments. Scanner 410 may comprise a PET/CT scanner capable of generating PET data and CT data associated with an object such as patient 415. Embodiments are not limited to the use of a single scanner to produce PET data and CT data.

Scanner 410 uses an energy-integrating CT detector to acquire CT data and applies conventional CT reconstruction algorithms to the acquired CT data to generate energy-integrating CT image 420. Energy-integrating CT image 420 is input to trained neural network 430 to generate simulated photon-counting CT image 440.

Linear attenuation coefficient map (i.e. “mu-map”) 450 is derived from simulated photon-counting CT image 440 as is known in the art. A mu-map provides attenuation coefficients of subject tissue and is typically used for attenuation correction of emission data such as PET data and single-photon-emission-computer-tomography (SPECT) data during image reconstruction.

Scanner 410 acquires PET data 470 using any suitable PET data acquisition protocol. PET imaging generates quantitative images which represent biological processes (e.g., glucose metabolism, receptor affinity) occurring within a patient. PET images can help doctors diagnose and stage diseases, plan treatment, and evaluate the effectiveness of treatment.

In PET imaging, a radiotracer is administered to a patient via intravenous injection, inhalation, oral ingestion or direct organ injection. The tracer experiences radioactive decay as it travels within the patient, generating positrons which eventually encounter electrons and are annihilated thereby. An annihilation produces two 511 keV photons which travel in approximately opposite directions.

A ring of detectors surrounds the patient, and a coincidence is identified when two of the detectors detect the arrival of two photons within a short time window indicating that the two photons arose from the same positron annihilation. Because the two “coincident” photons travel in approximately opposite directions, the locations of the two detector crystals determine a Line-of-Response (LoR) along which an annihilation may have occurred. PET data represents each detected annihilation as a LoR between two detector crystals. Time-of-flight (ToF) PET additionally measures the difference between the detection times of the two photons arising from the annihilation. This difference may be used to estimate a particular position along the LoR at which the annihilation event occurred.

PET data 470 may represent the detected coincidences as raw (i.e., list-mode) data and/or sinograms. List-mode data represents each coincidence using data specifying a LoR between two crystals, the time at which each photon of the annihilation reached each crystal, the photon energies, etc. A sinogram is a data array of the angle versus the displacement of the LoRs of each detected coincidence. A sinogram includes one row containing the LoR for a particular azimuthal angle φ. Each of these rows corresponds to a one-dimensional parallel projection of the tracer distribution at a different coordinate. A sinogram stores the location of the LoR of each coincidence such that all the LoRs passing through a single point in the volume trace a sinusoid curve in the sinogram.

PET reconstruction component 480 reconstructs three-dimensional PET image 490 from mu-map 460 and PET data 470 as is known in the art. PET reconstruction component 480 may reconstruct PET image 490 using algorithms such as filtered backprojection (FBP) and ordered subsets expectation maximization (OSEM), but embodiments are not limited thereto. Reconstruction may include any other suitable steps, such as subtraction of random coincidences and scatter coincidences from PET data 470, motion correction, and correction for system sensitivity.

PET data 470 and the CT data used to generate image 420 may be acquired substantially contemporaneously. For example, a PET imaging system of scanner 410 may be operated to acquire PET data 470 while patient 415 lies in a given position on a bed of scanner 410, and a CT imaging system of scanner 410 may be operated shortly thereafter to acquire CT data while patient 415 remains on the bed in the given position. Since the geometric transformation (if any) between coordinates of the PET imaging system and the CT imaging system is known, the CT data (and, as a result, mu-map 460) and PET data 470 may be easily spatially registered with one another.

FIG. 5 is a flow diagram of process 500 to train a neural network to generate a photon-counting CT image from an energy-integrating CT image according to some embodiments. Process 500 may be performed by any combination of hardware and software that is or becomes known. Program code embodying processes described herein may be stored by any non-transitory tangible medium, including a fixed disk, a volatile or non-volatile random-access memory, a DVD, a Flash drive, and a magnetic tape, and executed by any suitable processing unit, including but not limited to one or more microprocessors, microcontrollers, processor cores, and processor threads. Embodiments are not limited to the examples described below.

A plurality of energy-integrating CT images and corresponding photon-counting CT images are acquired at S510. An energy-integrating CT image and its corresponding photon-counting CT image may be acquired by scanning a portion of an object with an energy-integrating CT detector as described above and then scanning a same portion of the object with a photon-counting CT detector. The energy-integrating CT image is reconstructed from the data acquired by the energy-integrating CT detector and the corresponding photon-counting CT image is reconstructed from the data acquired by the photon-counting CT detector. Some or all of the energy-integrating CT images and corresponding photon-counting CT images may be acquired from one or more medical image repositories.

A single energy-integrating CT image may correspond to several photon-counting CT images. For example, a portion of an object may be scanned with an energy-integrating CT detector to acquire an energy-integrating CT image and the same portion may be scanned multiple times with a photon-counting CT detector to acquire multiple corresponding photon-counting CT images, where each scan with the photon-counting CT detector uses different energy thresholds. Generally, the photon-counting CT images acquired at S510 may represent many different sets of energy thresholds.

In some embodiments, each acquired photon-counting CT image is registered with its corresponding energy-integrating CT image at S520. Registration at S520 may be performed using any image registration technique that is or becomes known. Image registration may improve the effectiveness of the subsequent training steps of process 500. In this regard, a neural network is trained at S530 to generate a photon-counting CT image from an energy-integrating CT image based on the acquired photon-counting CT images and their corresponding energy-integrating CT images.

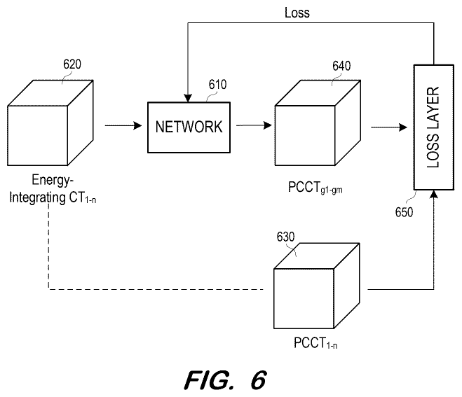

FIG. 6 illustrates training of neural network 610 to generate a simulated photon-counting CT image according to some embodiments. Network 610 is depicted as a supervised learning-compatible network. Network 610 may conform to the UNet or Pix2Pix architectures, for example. Network 610 may comprise any type of supervised or unsupervised learning-compatible network, algorithm, decision tree, etc. to receive image data and to output image data that is or becomes known.

Network 610 may comprise a plurality of layers of neurons which receive input, change internal state according to that input, and produce output depending on the input and internal state. The output of certain neurons is connected to the input of other neurons to form a directed and weighted graph. The weights as well as the functions that compute the internal states are iteratively modified during training.

The training data of FIG. 6 consists of N energy-integrating CT images 620 and N corresponding photon-counting CT images 630 acquired at S510. Each photon-counting CT image 630 is a “ground truth” associated with its corresponding energy-integrating CT image 620. Each of the N pairs of images 620 and 630 may be generated from data acquired by different detectors and may depict different subjects (i.e., patients).

During one example of network training, a batch of M energy-integrating CT images 620 is input to network 610. Network 610 operates according to its architecture and current hyperparameter values to generate a simulated photon-counting CT image 640 from each image 620 of the batch. Loss layer 650 calculates a loss based on differences between each of the M generated simulated photon-counting CT images 640 and its corresponding ground truth photon-counting CT image 630. The loss is back-propagated to network 610, which is modified to minimize the loss. Batches continue to be input and network 610 continues to be modified as described above until training is determined to be complete.

Once training is complete, trained network 610 may be deployed in a system such as system 300 or system 400 to generate simulated photon-counting CT images from input energy-integrating CT images. Trained network 610 may be deployed as a set of linear equations, executable program code, a set of hyperparameters defining a model structure and a set of corresponding weights, or any other executable representation of the mapping of input to output which was learned as a result of the training.

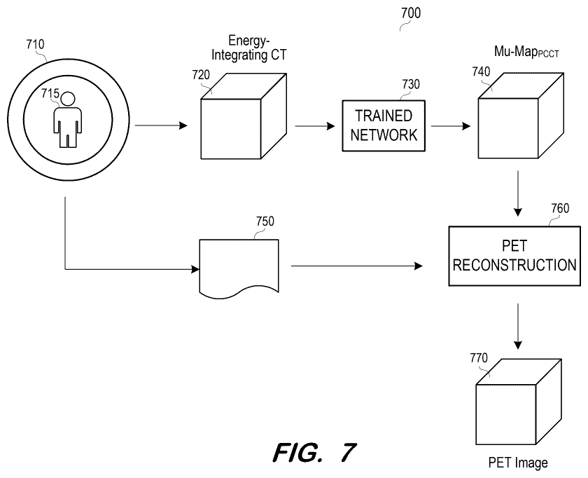

FIG. 7 is a block diagram of system 700 to generate a PET image based on a network-generated simulated mu-map according to some embodiments. Scanner 710 may comprise a PET/CT scanner as described above. Scanner 710 uses an energy-integrating CT detector to acquire CT data of patient 715 and CT reconstruction algorithms are applied to the acquired CT data to generate energy-integrating CT image 720. Energy-integrating CT image 720 is then input to trained neural network 730 to generate simulated mu-map 740. Simulated mu-map 740 is intended to simulate a mu-map determined from a CT image generated by scanning patient 715 with a photon-counting CT detector. Such a mu-map may provide more-accurate attenuation coefficients than a mu-map which is determined from energy-integrating CT image 720 using conventional techniques.

Scanner 710 acquires PET data 750 contemporaneously with the CT data. PET reconstruction component 760 reconstructs three-dimensional PET image 770 from simulated mu-map 740 and PET data 750. PET image 770 may exhibit less noise and more accurate quantification than a PET image which is reconstructed from PET data 750 and a mu-map determined from energy-integrating CT image 720 using conventional techniques.

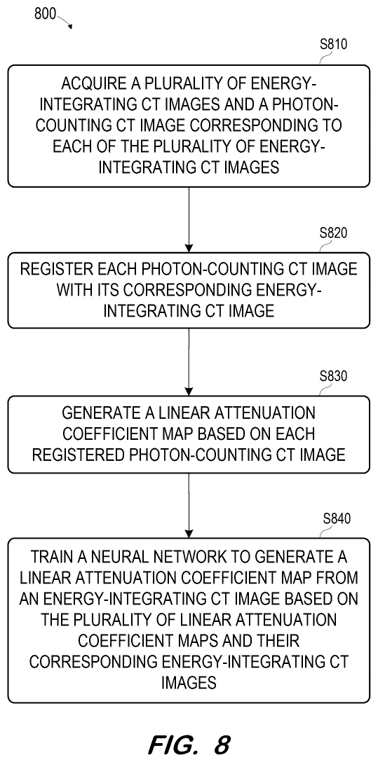

FIG. 8 is a flow diagram of process 800 to train a neural network to generate a simulated mu-map from an energy-integrating CT image according to some embodiments. A plurality of energy-integrating CT images and corresponding photon-counting CT images are acquired at S810 and registered to one another at S820 as described with respect to S510 and S520.

Next, at S830, a mu-map is generated from each of the registered photon-counting CT images. As is known in the art, generation of a mu-map at S830 may comprise converting the values of each voxel of a photon-counting CT image from Hounsfield Units to Attenuation Coefficients. A neural network is trained at S840 to generate a simulated mu-map from an energy-integrating CT image based on the mu-maps generated at S830 and their corresponding energy-integrating CT images.

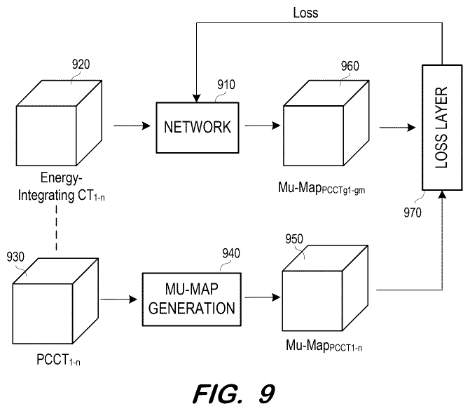

FIG. 9 illustrates training of neural network 910 to generate a simulated mu-map based on an energy-integrating CT image according to some embodiments. Network 910 may comprise any type of suitable neural network that is or becomes known.

FIG. 9 depicts N energy-integrating CT images 920 and N corresponding photon-counting CT images 930 acquired at S 810. N mu-maps 950 are generated by mu-map generation component 940 from corresponding ones of N photon-counting CT images 930. Accordingly, each of the N mu-maps is a “ground truth” associated with a corresponding energy-integrating CT image 920.

In one example of network training at S840, a batch of M energy-integrating CT images 920 is input to network 910. Network 910 generates a simulated mu-map 960 from each image 920 of the batch. Loss layer 970 calculates a loss based on differences between each of the M generated simulated mu-maps 960 and a corresponding ground truth mu-map 950. The loss is back-propagated to network 910 and the process repeats until training is complete. The resulting trained network 910 may be deployed in a system such as system 700 to generate simulated mu-maps from input energy-integrating CT images.

FIG. 9 illustrates training of neural network 910 to generate a simulated mu-map based on an energy-integrating CT image according to some embodiments. Network 910 may comprise any type of suitable neural network that is or becomes known.

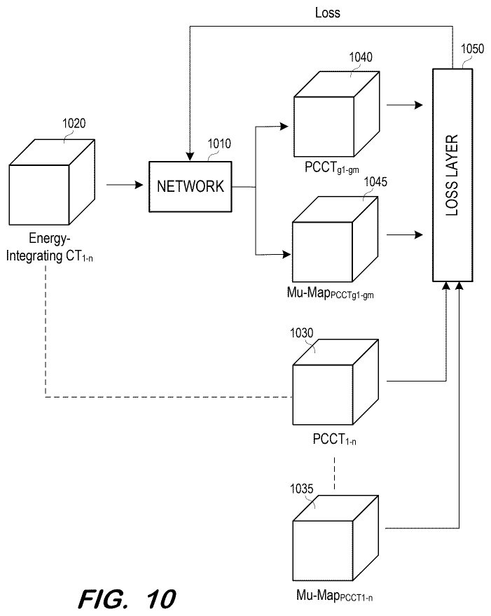

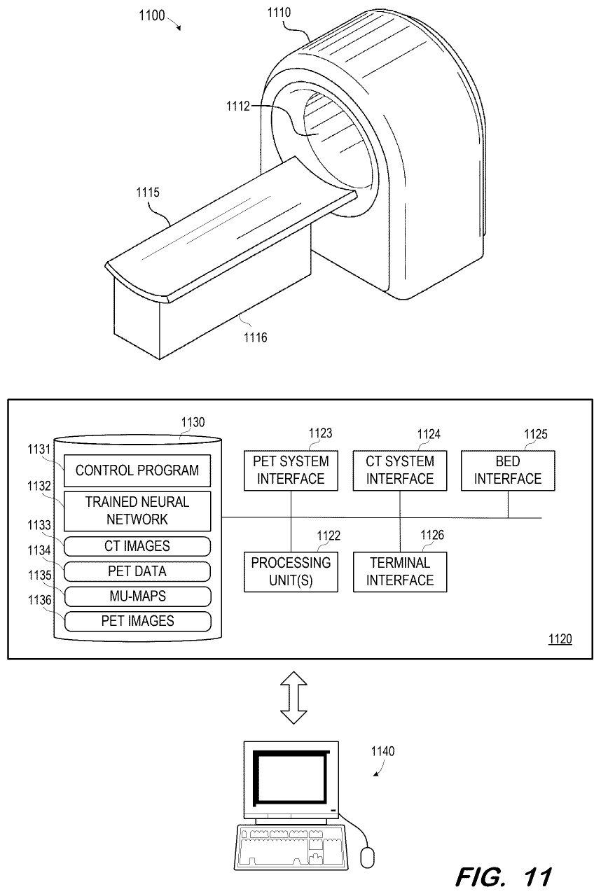

FIG. 10 depicts training of a neural network to generate both a simulated photon-counting CT image and a simulated mu-map from an energy-integrating CT image. The training data consists of N energy-integrating CT images 1020, N corresponding ground truth photon-counting CT images 1030 and N ground truth mu-maps 1035 generated from corresponding photon-counting CT images 1030. In each training epoch, a batch of M energy-integrating CT images 1020 is input to network 1010. Network 1010 generates a simulated photon-counting CT image 1040 and a simulated mu-map 1045 from each image 1020 of the batch. Loss layer 1050 calculates a loss based on differences between each of the M generated simulated photon-counting CT images 1040 and a corresponding ground truth photon-counting CT image 1030 and on differences between each of the M generated simulated mu-maps 1045 and a corresponding ground truth mu-map 1035. The loss is back-propagated to network 1010 and the process repeats until training is complete.

FIG. 11 illustrates PET/CT scanner 1100 to execute one or more of the processes described herein. Embodiments are not limited to scanner 1100 or to a multi-modality imaging system.

Scanner 1100 includes gantry 1110 defining bore 1112. As is known in the art, gantry 1110 houses PET imaging components for acquiring PET image data and CT imaging components for acquiring CT image data. The CT imaging components may include one or more x-ray tubes and one or more corresponding energy-integrating detectors as is known in the art. The PET imaging components may include any number or type of detectors including background radiation-emitting crystals and disposed in any configuration as is known in the art.

Bed 1115 and base 1116 are operable to move a patient lying on bed 1115 into and out of bore 1112 before, during and after imaging. In some embodiments, bed 1115 is configured to translate over base 1116 and, in other embodiments, base 1116 is movable along with or alternatively from bed 1115.

Movement of a patient into and out of bore 1112 may allow scanning of the patient using the CT imaging elements and the PET imaging elements of gantry 1110. Bed 1115 and base 1116 may provide continuous bed motion and/or step-and-shoot motion during such scanning according to some embodiments.

Control system 1120 may comprise any general-purpose or dedicated computing system. Accordingly, control system 1120 includes one or more processing units 1122 configured to execute program code to cause system 1120 to acquire image data and generate images therefrom, and storage device 1130 for storing the program code. Storage device 1130 may comprise one or more fixed disks, solid-state random-access memory, and/or removable media (e.g., a thumb drive) mounted in a corresponding interface (e.g., a Universal Serial Bus port).

Storage device 1130 stores program code of control program 1131. One or more processing units 1122 may execute control program 1131 to control CT imaging elements of scanner 1100 using CT system interface 1124 and bed interface 1125 to acquire CT data and to reconstruct energy-integrating CT images 1133 therefrom. One or more processing units 1122 may execute control program 1131 to, in conjunction with PET system interface 1123 and bed interface 1125, control hardware elements to inject a radiopharmaceutical into a patient, move the patient into bore 1112 past PET detectors of gantry 1110, and acquire PET data 1134 based on pulses generated by the PET detectors.

Trained neural network 1132 may be executable to generate a simulated photon-counting CT image from an acquired energy-integrating CT image. A mu-map 1135 may be generated from the simulated photon-counting CT image and used to reconstruct a PET image 1136 from PET data 1134. In some embodiments, neural network 1132 is executable to generate a simulated mu-map 1135 from an acquired energy-integrating CT image. Such a simulated mu-map 1135 may be used to reconstruct a PET image 1136 from PET data 1134.

PET images 1136 and CT images 1133 may be transmitted to terminal 1140 via terminal interface 1126. Terminal 1140 may comprise a display device and an input device coupled to system 1120. Terminal 1140 may display the received PET images 1136 and CT images 1134. Terminal 1140 may receive user input for controlling display of the data, operation of scanner 1100, and/or the processing described herein. In some embodiments, terminal 1140 is a separate computing device such as, but not limited to, a desktop computer, a laptop computer, a tablet computer, and a smartphone.

Each component of scanner 1100 may include other elements which are necessary for the operation thereof, as well as additional elements for providing functions other than those described herein. Each functional component described herein may be implemented in computer hardware, in program code and/or in one or more computing systems executing such program code as is known in the art. Such a computing system may include one or more processing units which execute processor-executable program code stored in a memory system.

Those in the art will appreciate that various adaptations and modifications of the above-described embodiments can be configured without departing from the claims. Therefore, it is to be understood that the claims may be practiced other than as specifically described herein.

What is claimed is:

1. A scanner to generate medical images, comprising:
a plurality of energy-integrating photon detectors to output electrical signals;
a display; and
a processing unit to:
generate a first image volume based on electrical signals output by the energy-integrating photon detectors;
input the first image volume to a network trained to generate a simulated photon-counting CT image from an energy-integrating CT image;
receive a first simulated photon-counting CT image generated by the network in response to the input first image volume; and
present the first simulated photon-counting CT image on the display.

2. The scanner of claim 1, further comprising:
a ring of positron emission tomography (PET) detectors to detect received photons,
the processing unit to:
generate a linear attenuation coefficient map based on the first simulated photon-counting CT image; and
reconstruct a PET image based on the detected received photons and the linear attenuation coefficient map.

3. The scanner of claim 2, wherein the network is trained based on a plurality of energy-integrating CT images and a plurality of photon-counting CT images, each of the plurality of photon-counting CT images corresponding to a respective one of the plurality of energy-integrating CT images.

4. The scanner of claim 3, wherein each of the plurality of photon-counting CT images depicts a same object as and is registered to its corresponding respective one of the plurality of energy-integrating CT images.

5. The scanner of claim 1, further comprising:
a ring of positron emission tomography (PET) detectors to detect received photons,
wherein the network is trained to generate a simulated photon-counting CT image and a simulated photon-counting CT linear attenuation coefficient map from an energy-integrating CT image,
the processing unit to:
receive a first simulated photon-counting CT linear attenuation coefficient map generated by the network in response to the input first image volume; and
reconstruct a PET image based on the detected received photons and the first simulated photon-counting CT linear attenuation coefficient map.

6. The scanner of claim 5, wherein the network is trained based on a plurality of energy-integrating CT images, a plurality of photon-counting CT images, and a plurality of photon-counting CT linear attenuation coefficient maps, each of the plurality of photon-counting CT images and the plurality of photon-counting CT linear attenuation coefficient maps corresponding to a respective one of the plurality of energy-integrating CT images.

7. The scanner of claim 6, wherein each of the plurality of photon-counting CT images depicts a same object as and is registered to its corresponding respective one of the plurality of energy-integrating CT images.

8. A scanner to generate medical images, comprising:
a plurality of energy-integrating photon detectors to output electrical signals;
a display; and
a processing unit to:
generate a first image volume based on electrical signals output by the energy-integrating photon detectors;
input the first image volume to a network trained to generate a simulated photon-counting CT linear attenuation coefficient map from an energy-integrating CT image;
receive a first simulated photon-counting linear attenuation coefficient map generated by the network in response to the input first image volume; and
present the first simulated photon-counting CT linear attenuation coefficient map on the display.

9. The scanner of claim 8, further comprising:
a ring of positron emission tomography (PET) detectors to detect received photons,
the processing unit to:
reconstruct a PET image based on the detected received photons and the first simulated photon-counting CT linear attenuation coefficient map.

10. The scanner of claim 9, wherein the network is trained based on a plurality of energy-integrating CT images and a plurality of photon-counting CT linear attenuation coefficient maps, each of the plurality of photon-counting CT linear attenuation coefficient maps corresponding to a respective one of the plurality of energy-integrating CT images.

11. The scanner of claim 10, wherein each of the plurality of photon-counting CT linear attenuation coefficient maps is generated from a photon-counting CT images which depicts a same object as and is registered to its corresponding respective one of the plurality of energy-integrating CT images.

12. A method comprising:
generating a first image volume based on electrical signals output by energy-integrating photon detectors;
inputting the first image volume to a network trained to generate a simulated photon-counting computed tomography (CT) image from an energy-integrating CT image;
receiving a first simulated photon-counting CT image generated by the network in response to the input first image volume; and
presenting the first simulated photon-counting CT image on a display.

13. The method of claim 12, further comprising:
generating a linear attenuation coefficient map based on the first simulated photon-counting CT image; and
reconstructing a PET image based on the received photons detected by a ring of positron emission tomography (PET) detectors and the linear attenuation coefficient map.

14. The method of claim 13, wherein the network is trained based on a plurality of energy-integrating CT images and a plurality of photon-counting CT images, each of the plurality of photon-counting CT images corresponding to a respective one of the plurality of energy-integrating CT images.

15. The method of claim 14, wherein each of the plurality of photon-counting CT images depicts a same object as and is registered to its corresponding respective one of the plurality of energy-integrating CT images.

16. The method of claim 12, wherein the network is trained to generate a simulated photon-counting CT image and a simulated photon-counting CT linear attenuation coefficient map from an energy-integrating CT image, further comprising:
receiving a first simulated photon-counting CT linear attenuation coefficient map generated by the network in response to the input first image volume; and
reconstructing a PET image based on the received photons detected by a ring of positron emission tomography (PET) detectors and the first simulated photon-counting CT linear attenuation coefficient map.

17. The method of claim 16, wherein the network is trained based on a plurality of energy-integrating CT images, a plurality of photon-counting CT images, and a plurality of photon-counting CT linear attenuation coefficient maps, each of the plurality of photon-counting CT images and the plurality of photon-counting CT linear attenuation coefficient maps corresponding to a respective one of the plurality of energy-integrating CT images.

18. The method of claim 17, wherein each of the plurality of photon-counting CT images depicts a same object as and is registered to its corresponding respective one of the plurality of energy-integrating CT images.
